(19) United States
(12) Patent Application Publication
(10) Pub. No.: US 2024/0366408 A1
(43) Pub. Date: Nov. 7, 2024

(54) IMPLANT, MORE PARTICULARLY STENT, AND METHOD OF PRODUCTION

(71) Applicant: Acandis GmbH, Pforzheim (DE)

(72) Inventors: Andreas Schüßler, Pfinztal (DE); Michael Büchert, Pforzheim (DE)

(21) Appl. No.: 18/293,519

(22) PCT Filed: Jul. 20, 2022
(86) PCT No.: PCT/EP2022/070323

§ 371 Date: Jan. 30, 2024

(30) Foreign Application Priority Data
Jul. 30, 2021 (DE) 10 2021 119 828.3
Nov. 4, 2021 (DE) 10 2021 128 698.0

(51) Int. Cl. A61F 2/90

(52) U.S. Cl. CPC A61F 2/90; A61F 2210/0014; A61F 2210/0076; A61F 2240/001; A61F 2250/0067; A61F 2250/0098

(57) ABSTRACT

A medical implant, more particularly a stent, having a tubular lattice structure which can be transferred from a radially expanded compressed state to a radially compressed expanded state and has lattice elements that delimit the cells of the lattice structure and have an element surface. In order to enlarge the element surface of each lattice element a polymer nanostructure is distributed over the entire element surface of the lattice element and adheres to it, and an antithrombogenic coating is provided and extends across the lattice element structure surface enlarged by means of the polymer nanostructure.

### CROSS REFERENCE TO RELATED PATENT APPLICATIONS

This is a U.S. national phase patent application of PCT/EP2022/070323 filed Jul. 20, 2022 which claims the benefit of and priority to German Patent Application No. 10 2021 128 698.0 filed on Nov. 4, 2021 and German Patent Application No. 10 2021 119 828.3 filed on Jul. 30, 2021, the entire contents of each of which are incorporated herein by reference.

### TECHNICAL FIELD

The invention relates to a medical implant, in particular a stent. The invention further relates to a method for the production of such an implant. An implant of the aforementioned type is known from EP 1 362 564 B1, for example.

### BACKGROUND

EP 1 362 564 B1 describes a self-expanding stent which is particularly suitable for the treatment of aneurysms. Specifically, the stent can be used to cover aneurysms in vessels in the brain.

The known stent or the known implant comprises a tubular mesh structure which can automatically dilate from a radially compressed state into a radially expanded state. In this regard, the mesh structure is formed from a plurality of interconnected mesh elements which delimit cells of the mesh structure.

In general, the overall mesh structure has an outer circumferential surface and an inner circumferential surface, wherein the mesh elements are disposed between the outer circumferential surface and the inner circumferential surface. Each individual mesh element has a respective element surface which is congruent with the outer circumferential surface and the inner circumferential surface. The surfaces of the respective mesh element which extend between the outer circumferential surface and the inner circumferential surface are also a component of the element surface. Consequently, each mesh element has an element surface which extends around the entire circumference of the mesh element.

Stents of the type mentioned above suffer from the disadvantage that in the installed state, they influence the flow of blood through a vessel, which on the one hand can lead to the formation of blood clots, and on the other hand can also complicate any integration of the stent into the natural blood vessel. In this regard, further developments of stents of this type were made in which the stents were coated with antithrombogenic substances. Medicine-releasing stents of this type have reduced the risk of the formation of blood clots or thromboses. However, the release of antithrombogenic substances of this type is time-limited, because the substance is released from the mesh structure until the mesh structure is essentially free of substance.

In this regard, there is a need for medical implants which can prevent the formation of blood clots over a long period and therefore can become better integrated into the organic structures.

### SUMMARY

The objective of the invention is therefore to further develop known medical implants in a manner such that their long-term effects are improved. A further objective of the invention is to provide a method for the production of a medical implant of this type.

In accordance with the invention, in respect of the medical implant, this objective is achieved by the subject matter as disclosed herein.

Thus, the invention is based on the concept of providing a medical implant, in particular a stent, with a tubular mesh structure which can be transposed from a radially compressed state into a radially expanded state. The mesh structure has mesh elements which delimit cells of the mesh structure. In addition, the mesh elements have an element surface. In accordance with the invention, in order to enlarge the element surface of each mesh element, a polymeric nanostructure is provided which is distributed over and adhered to the entire element surface of the mesh element. Furthermore, an antithrombogenic coating is provided which extends over the structural surface of the mesh element enlarged by the polymeric nanostructure.

The advantage of the invention lies in the fact that because of the enlargement of the element surface, compared with the prior art, significantly more antithrombogenic substance can be deposited on the mesh structure than is possible with implants from the prior art. This therefore means that larger quantities of a functional substance, in particular an antithrombogenic substance, can be deposited on the mesh structure so that the long-term efficacy of the antithrombogenic substance is improved.

The polymeric nanostructure enlarges the element surface of the mesh elements. The enlarged structural surface has the further advantage that, because of the structuring of the structural surface, endothelial cells can be deposited better or can form afresh better, so that the medical implant in accordance with the invention can become integrated into the organic environment very well and very rapidly.

The entirety of the polymeric nanostructure does not necessarily have to adhere to the entire element surface of the mesh network. In point of fact, the polymeric nanostructure is preferably only in contact with the element surface occasionally or at selected points. In the context of the present application, therefore, the element surface is the total surface of each mesh element without the polymeric nanostructure. The polymeric nanostructure is then applied to the element surface, wherein the polymeric nanostructure adheres to the element surface at selected points. The total surface of the mesh element resulting from this, i.e. the combination of the surface of the polymeric nanostructure and the regions of the element surface which are free of the polymeric nanostructure, forms the structural surface of the mesh element. Put simply, therefore, the element surface describes the surface of the mesh element without a polymeric nanostructure, whereas the structural surface describes the surface of the mesh element with the polymeric nanostructure.

The polymeric nanostructure may be formed from resorbable or non-resorbable polymers. In particular, the polymeric nanostructure may be produced from non-resorbable polymers based on polyurethane. Examples of resorbable biopolymers for forming the polymeric nanostructure are polylactides (poly-L-lactide (PLLA), polylactide-co-glycolide (PILCA)) or mixtures of these materials (copolymers). Further preferred materials are polycaprolactone (PCL), polylactide-co-caprolactone (PLCL), poly-D-lactide (PDLA) or poly-DL-lactide (PDLLA), as well as other compositions.

In a preferred embodiment of the invention, the polymeric nanostructure is hydrophobically bound to the element surface. The hydrophobic binding reduces the contact surface between the polymeric nanostructure and the element surface, so that a larger surface of the polymeric nanostructure is available for the deposition of endothelial cells and/or the antithrombogenic coating. Thus, the hydrophobic deposition results in a particularly large enlargement of the structural surface.

The antithrombogenic coating may be at least partially incorporated into the polymeric nanostructure, or may encase the polymeric nanostructure. It is also possible for the antithrombogenic coating to be at least partially interlinked with a surface of the polymeric nanostructure. The incorporation of the antithrombogenic coating into the polymeric nanostructure further enhances the durability or long-term efficacy of the antithrombogenic coating by slow diffusion of the substances at the surface.

Incorporation of the antithrombogenic coating into the polymeric nanostructure protects the antithrombogenic coating from abrasion which, for example, may arise from delivering the implant through a catheter. The antithrombogenic coating is effective for longer at the treatment site because of this. This is also the case when the antithrombogenic coating is at least partially interlinked with a surface of the polymeric nanostructure. Strong binding of the antithrombogenic coating to the polymeric nanostructure is advantageous in this respect.

In a preferred embodiment of the invention, the antithrombogenic coating has fibrin and an anticoagulant. It is also possible for the antithrombogenic coating to contain fibrin and an anticoagulant.

In particular, the anticoagulant may be heparin, which is covalently bound to the fibrin. Covalent binding of the heparin to the fibrin ensures that a particularly stable antithrombogenic coating is formed. In this regard, both fibrin and also heparin are effective antithrombogenically. The two substances complement each other in this respect and thereby have a particularly good antithrombogenic action.

In a preferred embodiment of the invention, the polymeric nanostructure is formed by polymer droplets which are deposited on and distributed over the element surface of the mesh element. Thus, for example, at least six polymer droplets may be deposited on a surface area of 16 μm², wherein the deposit is preferably hydrophobic. If, for example, each polymer droplet covers an area of approximately 0.66 μm² of the element surface in this situation, this covered surface is enlarged to a local structural surface area of approximately 2.2 μm². This arises from the near-spherical surface of the polymer droplets. An element surface area of 16 μm² is therefore enlarged to a structural surface area of 25.23 μm² with six hydrophobically deposited droplets. This corresponds to a surface area enlargement between the element surface and the structural surface of 158%.

In a preferred embodiment of the invention, the polymer droplets have a respective diameter of at most 1500 nm, in particular at most 1250 nm, in particular at most 1000 nm, in particular at most 750 nm, in particular at most 500 nm, in particular at most 400 nm, in particular at most 250 nm, in particular at most 100 nm, in particular at most 50 nm. It is possible for the polymer droplets to have different diameters. However, the aforementioned values for the diameters constitute an average of the diameters of all of the polymer droplets. Usually, the polymer droplets form a spherical surface which is only interrupted in a small segment because there, the polymer droplet is hydrophobically bound to the element surface. The smaller the diameter of the polymer droplet, then the larger is the enlargement of the structural surface area of the mesh element which is composed of the surface area of the polymer droplet and the element surface area of the mesh element.

In general, the polymeric nanostructure preferably forms a porous bonding agent on the element surface of the mesh element. The bonding agent is described as porous because the polymeric nanostructure does not adhere to the entire element surface, but only in places or at selected points. When the polymeric nanostructure is formed by polymer droplets, then free regions of the element surface of the mesh element are present between the individual droplets. These free regions are also part of the structural surface.

It is also possible for the polymeric nanostructure to be formed by a nano-nonwoven produced from polymer fibres which extend completely around the mesh element. The nano-nonwoven maybe produced by electrospinning, for example. With such a polymeric nanostructure, individual fibres are linked together or intersect each other chaotically and in this manner form a nonwoven. The nonwoven sheaths the element surface of each mesh element. However, because the individual fibres are only in contact with the element surface of the mesh element in places or at selected points, in the context of the present application, such a polymeric nanostructure is also described as a porous bonding agent.

The antithrombogenic coating which extends over the polymeric nanostructure may in particular be bonded to the nano-nonwoven produced from polymer fibres. In particular, the antithrombogenic substance of the antithrombogenic coating may be at least partially incorporated into the nano-nonwoven. This produces a particularly effective long-term substance release because the nano-nonwoven resembles a sponge and can accommodate a comparatively large quantity of the antithrombogenic coating.

Independently of the type of polymeric nanostructure, in particular independently of whether the polymeric nanostructure is formed from polymer droplets and/or from a nano-nonwoven, advantageously, with the polymeric nanostructure, the size of the structural surface area of the mesh element is enlarged by at least 150%, in particular at least 200%, in particular at least 250%, in particular at least 300%, in particular at least 400%, in particular at least 500% with respect to the size of the element surface. In other words, then, the surface area of the mesh element which is available for the deposition of the antithrombogenic coating is considerably enlarged by applying the polymeric nanostructure between the element surfaces of the mesh element and the antithrombogenic coating to obtain a substantial enlargement of the surface to which the antithrombogenic coating can bind.

It has been shown that the enlargement of the structural surface with respect to the element surface has a considerable influence on the efficacy of the antithrombogenic substance, in particular in terms of time. The enlargement of the structural surface area is particularly advantageous having regard to the integration of the implant into the surrounding biological tissue. By means of the enlargement of the element surface area, i.e. the surface area of the bare mesh element, by depositing the polymeric nanostructure, in practice and in addition, endothelial cells can be deposited effectively onto the mesh elements. This leads to a particularly rapid endothelialization of the implant, whereupon it integrates well into the natural tissue and therefore turbulence, which can later lead to a formation of blood clots, is avoided.

Preferably, the element surface may be surface treated, in particular electropolished or mechanically polished. Despite the smooth or polished element surface, the polymeric nanostructure surprisingly adheres well to the element surface.

In a further preferred embodiment of the implant in accordance with the invention, the mesh element has a core layer and a shell layer. The core layer may be a radiopaque material, in particular platinum or a platinum alloy, and the shell layer may include or consist of a superelastic material, in particular a nickel-titanium alloy. Essentially, therefore, the mesh element may be formed by a composite material, in which the core layer ensures improved radiopacity of the implant and the shell layer provides the implant with particularly high elastic properties. The superelastic material may in particular be formed by a shape memory metal, for example a nickel-titanium alloy. Materials of this type have a tendency to resume a previously set shape when the material reaches a specific temperature.

The mesh element, in particular the shell layer of the mesh element, may also have a titanium oxynitride layer which forms the element surface. Such a titanium oxynitride layer may be produced by heat treating the implant in a salt bath. The advantage of such a titanium oxynitride layer is that a proportion of the nickel in the material of the shell layer collects in the region of the inside of the titanium oxynitride layer, i.e. facing the core layer. On the outer surface, namely the element surface, therefore, there is hardly any nickel, whereupon what is known as nickel release is reduced. The nickel release describes the capability of implants to release nickel into the human body. As low a release as possible is desirable in order to avoid allergic reactions.

In accordance with a further embodiment of the invention, at least one medicine is incorporated into the polymeric nanostructure, in particular into the nano-nonwoven. Examples of suitable medicines are therapeutic substances for minimising restenosis, for healing diseased sections of vessels and/or for restricting cell growth. Other medicines may also be used. Because the medicine is incorporated into the polymeric nanostructure, for example held between the individual filaments of a nano-nonwoven, it is slowly released over a long period of time and therefore provides a long-lasting effect.

In general, the mesh structure may have a superelastic material, for example a nickel-titanium alloy such as nitinol. The mesh structure may in particular be self-expandable. However, it is also possible for the mesh structure to comprise another material, for example a cobalt-chromium alloy. Consequently, the mesh structure may also be balloon-expandable. In other words, an expansion of the mesh structure may be carried out by means of a balloon which is disposed on a catheter, for example.

Furthermore, in the context of the present application, a method for the production of an implant as described above is disclosed and claimed. In the method in accordance with the invention, the following steps are carried out:
a. providing a mesh structure with mesh elements,
b. applying a high voltage electrical field between the mesh structure and an emitter electrode,
c. spraying the mesh structure with a polymer solution, wherein the polymer solution has a proportion of at most 3 in particular at most 2%, in particular at most 1% of dissolved polymer, and
d. coating the mesh structure with an antithrombogenic coating.

### DESCRIPTION OF DRAWINGS

The invention will now be described in more detail with the aid of exemplary embodiments and with reference to the accompanying diagrammatic drawings, in which

FIG. 1 shows a cross sectional view of a mesh element of a tubular mesh structure of a medical implant in accordance with the invention according to a preferred exemplary embodiment prior to application of the polymeric nanostructure;

FIG. 2 shows a cross sectional view of the mesh element in accordance with FIG. 1 after application of the polymeric nanostructure;

FIG. 3 shows a cross sectional view of a mesh element of a tubular mesh structure of a medical implant in accordance with the invention according to a further preferred exemplary embodiment prior to the application of the polymeric nanostructure;

FIG. 4 shows a cross sectional view of the mesh element in accordance with FIG. 3 after application of the polymeric nanostructure;

FIG. 5 shows a scanning electron microscope image of the mesh element in accordance with FIG. 2:

FIG. 6 shows a scanning electron microscope image of the mesh element in accordance with FIG. 2; and

FIG. 7 shows a cross sectional view of a polymer droplet of the polymeric nanostructure of the medical implant in accordance with the invention according to a preferred exemplary embodiment.

### DESCRIPTION OF AN EMBODIMENT

The accompanying drawings each show details of a medical implant which is preferably used as a stent for insertion into blood vessels. The implant has a tubular mesh structure, i.e. a mesh structure which essentially forms a framework of a wall of a tube. The tubular shape is therefore not completely closed in its circumferential surface, but has mesh openings. In general, the mesh structure can be transposed from a radially compressed state into a radially expanded state. The implant or its mesh structure can therefore be guided in a narrow form through a catheter to the treatment site, whereupon the mesh structure is deployed at the treatment site. Preferably, the deployment is automatic. In other words, the mesh structure is preferably self-expandable.

The mesh structure comprises a plurality of mesh elements 10 which delimit cells of the mesh structure. The mesh elements 10 may be formed by webs 11. When the mesh elements 10 are formed by webs 11, the mesh structure is preferably produced by cutting out of a solid tubular material. The webs are thus connected to each other in one piece, so that overall, the mesh structure forms a one-piece component.

Alternatively, the mesh elements 10 may also be formed by wires 12. The wires 12 are preferably braided or woven together in order to form a mesh structure in this manner. The wires 12 cross over and under each other in a regular manner, wherein different interlacing patterns may be produced, Thus, for example, one wire 12 may cross over two further wires 12 and then pass under just one wire 12. Other patterns may be envisaged.

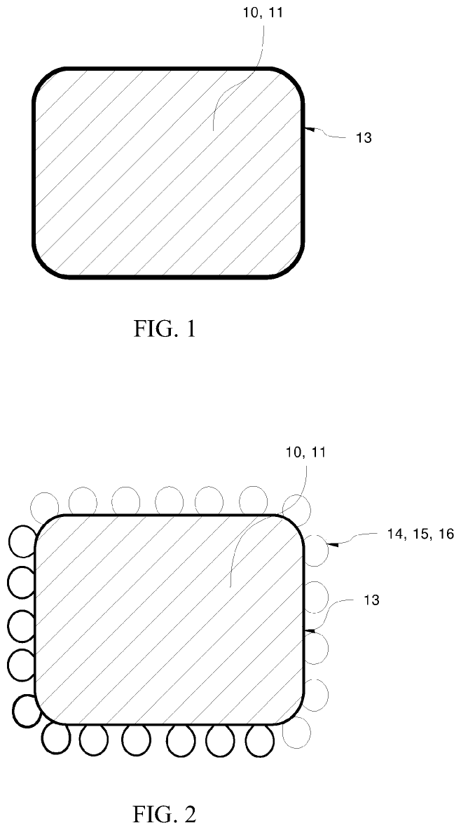

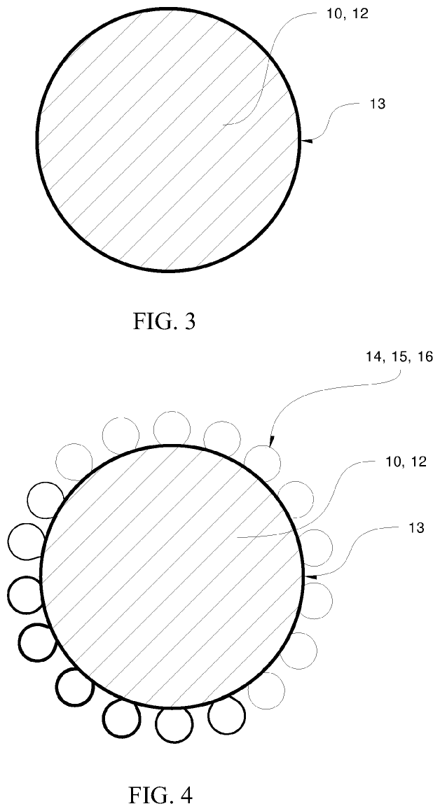

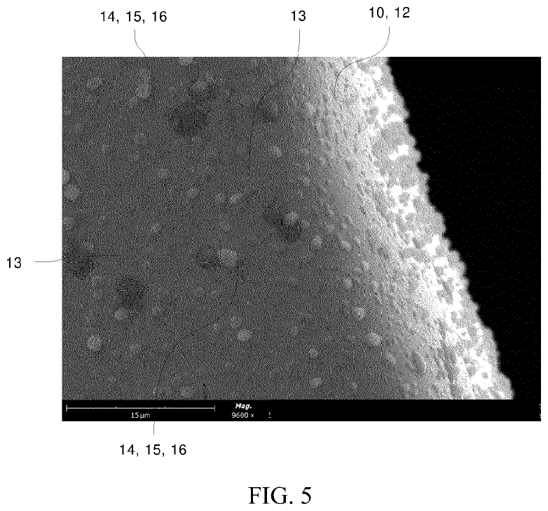

FIGS. 1 and 2 show a mesh element which is preferably configured as a web 11. In this regard, the mesh element 10 essentially has a rectangular cross sectional contour with rounded edges. The rounding of the edges may, for example be produced by means of an etching process. FIGS. 3 and 4, on the other hand, show a mesh element 10 which is formed as a wire 12 and in this regard has a circular cross section.

The mesh elements 10 have an element surface 13. The element surface 13 is substantially bare in the embodiments in accordance with FIGS. 1 and 3. It can be seen that the element surface forms a substantially smooth surface. Thus, FIGS. 1 and 3 respectively show the mesh element 10 in the uncoated state.

FIGS. 2 and 4 show the mesh elements 10 which include a polymeric nanostructure. In these exemplary embodiments, the polymeric nanostructure 14 is respectively formed by a plurality of polymer droplets 15. The polymer droplets 15 may be applied to the element surface 13 in a spraying process. It can be seen that the polymeric nanostructure 14 or the polymer droplets 15 cause the surface of the mesh element 10 to be enlarged. In fact, the polymer droplets occupy a portion of the element surface 13 to which they adhere. The polymer droplets 15 themselves, however, have an additional, substantially spherical segment-shaped surface which, together with the gaps between the polymer droplets 15, form a structural surface 16 of the mesh element 10. The structural surface 16 is significantly larger than the element surface 13. In particular, a three-dimensional structural surface 16 is formed from the substantially two-dimensional element surface 13 by depositing the polymer droplets 15.

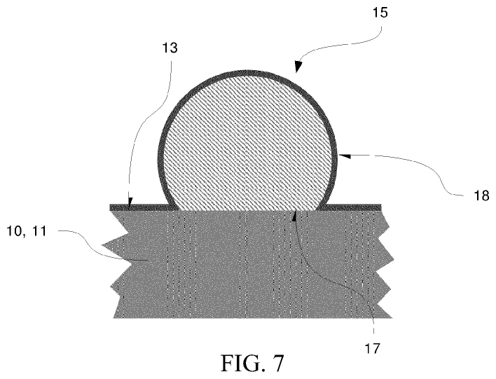

FIG. 7 shows the degree of surface enlargement with the aid of a polymer droplet 15. The polymer droplet 15 adheres, preferably hydrophobically, to the element surface 13 of the mesh element 10. This (two-dimensional) contact surface 17 is comparatively small. However, the polymer droplet 15 takes tip the shape of a (three-dimensional) segment of a sphere, wherein the spherical segment is larger than a hemisphere. The free droplet surface 18 is many times larger than the contact surface 17. In this manner, by spraying the element surface 13 with the polymeric nanostructure 14, it is possible to provide an enlarged structural surface 16 which is at least 50%, in particular at least 100%, in particular at least 150%, in particular at least 200%, in particular at least 300%, in particular at least 400% larger than the element surface. In other words, the structural surface 16 is at least one and a half times larger than the element surface 13.

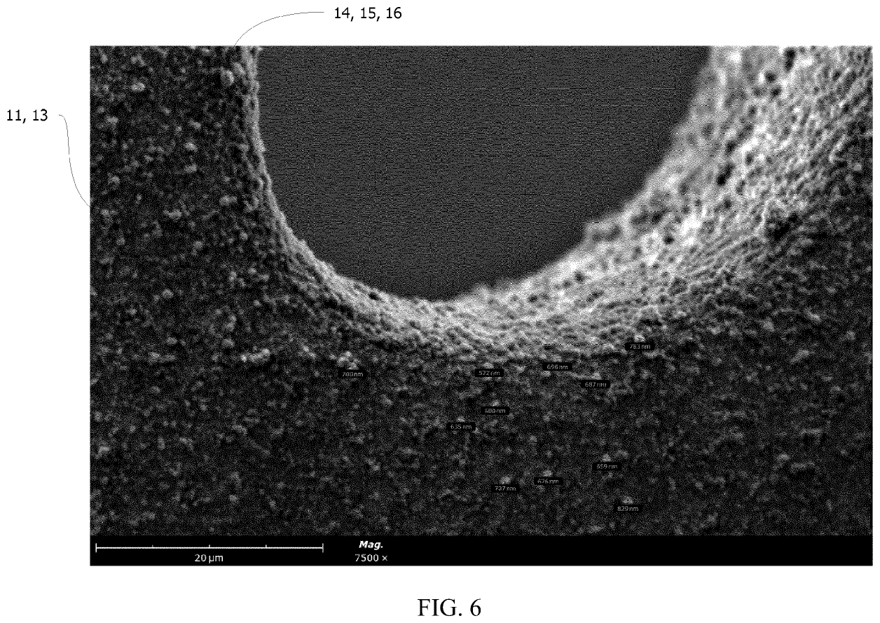

FIG. 5 shows a scanning electron microscope image of a slightly shaped structural surface 16 on the mesh element 10 or web 11 in accordance with FIG. 2, It can be seen that by spraying the polymeric nanostructure 14, an essentially porous layer of bonding agent is formed. The porosity is generated because the polymeric nanostructure 14 does not form a continuous layer which completely covers the element surface 13. Rather, the polymeric nanostructure 14 is formed from many polymer droplets 15 which are distributed over the element surface 13. The distributed arrangement of the polymer droplet 15 itself provides for the significant enlargement of the structural surface 16 with respect to the element surface 13.

The enlargement of the structural surface 16 with respect to the element surface 13 is even more significant when the density of the polymer droplets 15 is increased further. FIG. 6 shows such a further increased enlargement of the structural surface 16. Specifically, FIG. 6 shows a scanning electron microscope image of a section of a mesh structure produced from a plurality of mesh elements 10 which in the present case are configured as webs 11. The webs 11 are connected together into one piece at connecting points. FIG. 6 shows a portion of such a connecting point at which two webs 11 meet each other at an angle or merge. The element surface 13 is coated with the polymeric nanostructure 14 which almost completely covers the element surface 13 and forms the structural surface 16. The polymeric nanostructure 14 comprises or consists of a plurality of polymer droplets 15 which can be generated by spraying a polymer solution onto the element surface 13. The density of the polymer droplets 13 is selected so as to be high enough for at least a major portion of the polymer droplets 13 to be in contact with each other. Here, the droplet shape of the polymer droplets 13 increases the adhesive surface shown as the structural surface 16 and which is available for adhesion of the antithrombogenic coating.

The polymeric nanostructure 14 essentially acts as a bonding agent so that a further antithrombogenic nano-coating, not shown in the drawings, can adhere well and in large quantities to the mesh element 10. The antithrombogenic coating may in particular contain a combination of fibrin and an anticoagulant. Heparin or albumin are particularly effective anticoagulants. A combination of fibrin and heparin is particularly preferred in this case.

It is not just pure enlargement of the structural surface 16 with respect to the element surface 13 which is of advantage in the invention. The shape and structure of the polymer droplets 15 may be used to improve the deposition of the antithrombogenic coating onto the structural surface 16. Essentially, the shape of the polymer droplets 15, which form a segment of a sphere which is larger than a hemisphere, also brings about binding between the antithrombogenic coating and the structural surface 16 which can be described as at least partially interlocking. The portion of the droplet surface 18 which goes beyond the hemispherical shape and reaches to the contact surface 17 essentially forms a kind of undercut for the adhesion of the antithrombogenic coating. In this manner, the antithrombogenic coating is stable over particularly long periods and in particular is also resistant to abrasion which may occur when delivering an implant through a catheter.

As a result, then, the implant in accordance with the invention is particularly stable and effective over long periods, which leads to significantly improved therapeutic outcomes.

Instead of a polymeric nanostructure 14 which is formed from polymer droplets 15, forming the polymeric nanostructure 14 from a nano-nonwoven may also be envisaged. In this regard, polymer materials are applied to the element surface 13, for example by means of an electrospinning process, wherein individual fibres intersect in a chaotic manner. However, an air space remains between the individual fibres of the nano-nonwoven, in which air space the antithrombogenic coating can be deposited or can be anchored to the polymeric nano-nonwoven. Essentially, the nano-nonwoven thus acts like a sponge which takes in the antithrombogenic coating and slowly releases it at the treatment site so that the antithrombogenic coating can deploy its antithrombogenic action over a long period.

### REFERENCE NUMERALS

10 mesh element
11 web
12 wire
13 element surface
14 polymeric nanostructure
15 polymer droplet
16 structural surface
17 contact surface
18 droplet surface

1-14. (canceled)

15. A medical implant comprising a tubular mesh structure which can be transposed from a radially compressed state into a radially expanded state, and having a mesh element which delimits cells of the mesh structure and an element surface, wherein in order to enlarge the element surface of each mesh element, a polymeric nanostructure is provided which is distributed over and adhered to an entire element surface of the mesh element, wherein an antithrombogenic coating is provided which extends over a structural surface of the mesh element enlarged by the polymeric nanostructure.

16. The implant as claimed in claim 15, wherein the polymeric nanostructure is hydrophobically bound to the element surface.

17. The implant as claimed in claim 15, wherein the antithrombogenic coating is at least partially incorporated into the polymeric nanostructure or is at least partially interlinked with a surface of the polymer nanostructure.

18. The implant as claimed in claim 15, wherein the antithrombogenic coating has fibrin and an anticoagulant.

19. The implant as claimed in claim 18, wherein the anticoagulant is heparin which is covalently bound to the fibrin.

20. The implant as claimed in claim 15, wherein the polymeric nanostructure is formed by polymer droplets which are deposited on and distributed over the element surface of the mesh element.

21. The implant as claimed in claim 20, wherein the polymer droplets have a respective diameter of at most 1500 nm.

22. The implant as claimed in claim 15, wherein the polymeric nanostructure is formed by a nano-nonwoven produced from polymer fibres which extend completely around the mesh element.

23. The implant as claimed in claim 15, wherein the structural surface area enlarges the element surface by at least 150% with respect to the size of the element surface area.

24. The implant as claimed in claim 15, wherein the element surface is surface-treated by electropolishing or mechanically polishing.

25. The implant as claimed in claim 15, wherein the mesh element has a core layer and a shell layer, wherein the core layer includes a radiopaque material of a platinum or a platinum alloy, and the shell layer includes a superelastic material of a nickel-titanium alloy.

26. The implant as claimed in claim 15, wherein the mesh element has a titanium oxynitride layer which forms the element surface.

27. The implant as claimed in claim 15, wherein at least one medicine for minimizing restenosis, for healing diseased sections of vessels, or for restricting cell growth is incorporated into the polymeric nanostructure.

28. A method for the production of an implant as claimed in claim 15, the method comprising steps of:
a. providing the mesh structure with the mesh element,
b, applying a high voltage electrical field between the mesh structure and an emitter electrode,
c. spraying the mesh structure with a polymer solution, wherein the polymer solution has a proportion of at most 3% of dissolved polymer, and
d. coating the mesh structure with the antithrombogenic coating.
